(19) United States
(12) Patent Application Publication
(10) Pub. No.: US 2005/0287570 A1
(43) Pub. Date: Dec. 29, 2005

(54) Probe arrays for expression profiling of rat genes

(75) Inventor: William M. Mounts, Andover, MA (US)

Correspondence Address: NIXON PEABODY LP, 401 9TH STREET, N.W., SUITE 900, WASHINGTON, DC, 20004, US

(73) Assignee: Wyeth, Madison, NJ (US)

(21) Appl. No.: 11/136,527

(22) Filed: May 25, 2005

Related U.S. Application Data
(60) Provisional application No. 60/574,294, filed on May 26, 2004.

(51) Int. Cl. C12Q 1/68; C07H 21/04

(52) U.S. Cl. 435/6; 536/24.3

(57) ABSTRACT

The present invention provides probe arrays for expression profiling of rat genes. Each probe array comprises a plurality of probes, each of which is directed to a rat gene that encodes a sequence selected from SEQ ID NOs: 1-8,192. Suitable probes for the present invention include polynucleotides that can hybridize under stringent or nucleic acid array hybridization conditions to the RNA transcripts, or the complements thereof, of the corresponding rat genes. Suitable probes also include antibodies or other protein-binding molecules that can bind to the protein products of the corresponding rat genes. In one embodiment, a probe array of the present invention includes one or more probes, each of which is directed to a rat gene that encodes a sequence selected from SEQ ID NOs: 7622 and 8084-8124.

This application claims the benefit of, and incorporates by reference in its entirety, U.S. Provisional Application Ser. No. 60/574,294 filed May 26, 2004. All materials recorded in the compact discs labeled “Copy 1” and “Copy 2” are incorporated herein by reference in their entireties. Each of the compact discs includes the following files: “Table 1” (583 KB, created Apr. 12, 2004), “Table 2” (81 KB, created Apr. 12, 2004), “Table 4” (653 KB, created Apr. 12, 2004), “Table 5” (77 KB, created Apr. 12 , 2004), “Table 6” (5,576 KB, created Apr. 13, 2004), “Table 8” (10,321 KB, created Apr. 13, 2004), and “Sequence Listing.ST25.txt” (75,380 KB, created May 24, 2005).

### TECHNICAL FIELD

The present invention relates to probe arrays and methods of using the same for expression profiling of rat genes.

### BACKGROUND

The rat is one of the most widely used animal models of human disease. Many human disease genes have rat orthologs. Numerous rat models have been established for studying human diseases. Examples of these diseases include cancers, diabetes, arthritis, asthma, neurodegenerative diseases, hypertension, stroke, cardiovascular diseases, psychiatric stress, depression, and behavioral disorders. Moreover, rats are routinely used to demonstrate therapeutic efficacy or assess toxicity of novel drugs. Therefore, the rat is an indispensable platform for biomedical research and drug development.

Nucleic acid arrays, such as DNA microarrays, allow for simultaneous detection of a large number of genes. The use of nuclear acid arrays has significantly accelerated the process of drug discovery and development. Commercial rat nucleic acid arrays include the Rat Genome U34 arrays manufactured by Affymetrix. Each Rat Genome U34 array consists of probes for over 7,000 rat genes and 17,000 rat expression sequence tags (ESTs). Rat nucleic acid arrays have been employed in a variety of scientific disciplines such as toxicology, neurobiology, and physiology.

### SUMMARY OF THE INVENTION

The present invention features probe arrays for expression profiling of rat genes. The present invention also features methods of using these arrays for the identification and validation of drug targets and for the assessment and selection of drugs.

In one aspect, the probe arrays of the present invention are nucleic acid arrays. Each nucleic acid array includes polynucleotide probes capable of hybridizing under stringent or nucleic acid array hybridization conditions to the RNA transcripts, or the complements thereof, of corresponding rat genes. In another aspect, the probe arrays of the present invention are protein arrays. Each protein array includes antibodies or other molecules that can bind to the protein products of corresponding rat genes.

In one embodiment, a probe array of the present invention includes at least 10, 50, 100, 1,000, 2,000, 3,000, 4,000, or more probes, each of which is directed to a different respective rat gene that encodes a parent sequence selected from SEQ ID NOs: 1-4,096. In another embodiment, a probe array of the present invention includes at least 10, 50, 100, 1,000, 2,000, 3,000, 4,000, or more probes, each of which is directed to a different respective rat gene that encodes a tiling sequence selected from SEQ ID NOs: 4,097-8,192. In many cases, a substantial portion of all probes that are stably attached to a probe array of the present invention is probes for rat genes.

In yet another embodiment, a probe array of the present invention includes at least 1, 2, 3, 4, 5, 6, 7, 8, 9, 10, 20, 30, 40, or more probes, each of which is directed to a different respective rat gene that encodes a tiling sequence selected from the group consisting of WAN00OGR4, WAN00OGRF, WAN00OGS3, WAN00OGS4, WAN00OGS5, WAN00OGS6, WAN00OGS7, WAN00OGS8, WAN00OGS9, WAN00OGSA, WAN00OGSB, WAN00OGSC, WAN00OGSD, WAN00OGSE, WAN00OGSF, WAN00OGSG, WAN00OGSH, WAN00OGSI, WAN00OGSJ, WAN00OGSK, WAN00OGSL, WAN00OGSM, WAN00OGSN, WAN00OGSO, WAN00OGSP, WAN00OGSQ, WAN00OGSR, WAN00OGSS, WAN00OGST, WAN00OGSU, WAN00OGSV, WAN00OGSW, WAN00OGSX, WAN00OGSY, WAN00OGSZ, WAN00OGT0, WAN00OGT1, WAN00OGT2, WAN00OGT3, WAN00OGT4, WAN00OGT5, and WAN00OGT6.

In still yet another embodiment, a probe array of the present invention includes at least 1, 2, 3, 4, 5, 6, 7, 8, 9, or 10 probes, each of which is directed to a different respective rat gene that encodes a tiling sequence selected from the group consisting of WAN00OGR4, WAN00OGSD, WAN00OGSE, WAN00OGSH, WAN00OGSK, WAN00OGSN, WAN00OGSP, WAN00OGSQ, WAN00OGS4, and WAN00OGT4.

In still another embodiment, a probe array of the present invention includes at least one probe which is directed to a rat gene that encodes a tiling sequence selected from WAN00OGS4 and WAN00OGT4.

Multiple probes can be used for the detection of the same rat gene. In one example, a probe array of the present invention includes at least 25 probes for each rat gene being investigated. In another example, a probe array of the present invention includes each and every polynucleotide probe selected from SEQ ID NOs: 8,193-174,863, or the complement thereof.

The present invention also features methods of using probe arrays for detecting or monitoring gene expression in rat cells. In one embodiment, the probe array employed is a nucleic acid array, and the method comprises preparing a nucleic acid sample from rat cells and hybridizing the nucleic acid sample to the nucleic acid array. The hybridization signals are indicative of the expression levels of the corresponding genes in the rat cells. In another embodiment, the probe array employed is a protein array, and the method comprises preparing a protein sample from rat cells and contacting the protein sample with the protein array. The levels of binding to the protein array are indicative of the expression levels of the corresponding genes in the rat cells.

The present invention further features methods for identifying or evaluating agents that can modulate the expression of rat genes. In one embodiment, the methods include contacting an agent with rat cells, preparing a nucleic acid sample from the rat cells, and hybridizing the nucleic acid sample to a nucleic acid array of the present invention to determine if the agent is capable of modulating the expression of any rat gene. In many cases, the agents thus identified can modulate the expression of rat orthologs or homologs of human drug target genes. These human drug target genes encode, without limitation, kinases, phosphatases, proteases, G-protein coupled receptors, nuclear hormone receptors, or ion channels.

In addition, the present invention features polynucleotide or polypeptide collections. In one embodiment, a polynucleotide collection of the present invention includes at least one isolated polynucleotide comprising or consisting of a sequence selected from SEQ ID NOs: 1-8,192, or the full complement thereof. In another embodiment, a polynucleotide collection of the present invention includes at least one isolated polynucleotide comprising or consisting of a sequence selected from WAN00OGR4, WAN00OGRF, WAN00OGS3, WAN00OGS4, WAN00OGS5, WAN00OGS6, WAN00OGS7, WAN00OGS8, WAN00OGS9, WAN00OGSA, WAN00OGSB, WAN00OGSC, WAN00OGSD, WAN00OGSE, WAN00OGSF, WAN00OGSG, WAN00OGSH, WAN00OGSI, WAN00OGSJ, WAN00OGSK, WAN00OGSL, WAN00OGSM, WAN00OGSN, WAN00OGSO, WAN00OGSP, WAN00OGSQ, WAN00OGSR, WAN00OGSS, WAN00OGST, WAN00OGSU, WAN00OGSV, WAN00OGSW, WAN00OGSX, WAN00OGSY, WAN00OGSZ, WAN00OGT0, WAN00OGT1, WAN00OGT2, WAN00OGT3, WAN00OGT4, WAN00OGT5 and WAN00OGT6, or the full complement thereof. In still another embodiment, a polynucleotide collection of the present invention includes at least one isolated polynucleotide comprising or consisting of a sequence selected from WAN00OGR4, WAN00OGSD, WAN00OGSE, WAN00OGSH, WAN00OGSK, WAN00OGSN, WAN00OGSP, WAN00OGSQ, WAN00OGS4, and WAN00OGT4, or the full complement thereof. In yet another embodiment, a polynucleotide collection of the present invention includes at least one isolated polynucleotide comprising or consisting of a sequence selected from WAN00OGS4 and WAN00OGT4, or the full complement thereof. In still yet another embodiment, a polypeptide collection of the present invention comprises an isolated protein product of a rat gene that encodes a sequence selected from SEQ ID NOs: 1-8,192.

Other features, objects, and advantages of the present invention are apparent in the detailed description that follows. It should be understood, however, that the detailed description, while indicating preferred embodiments of the invention, is given by way of illustration only, not limitation. Various changes and modifications within the scope of the invention will become apparent to those skilled in the art from the detailed description.

### DETAILED DESCRIPTION

The present invention features probe arrays and methods of using the same for expression profiling of rat genes. In one embodiment, a probe array of the present invention includes a plurality of probes, each of which is directed to a rat gene that encodes a parent sequence selected from SEQ ID NOs: 1-4,096. In another embodiment, a probe array of the present invention includes a plurality of probes, each of which is directed to a rat gene that encodes a tiling sequence selected from SEQ ID NOs: 4,097-8,192. The probes employed in the present invention can be polynucleotides that can hybridize under stringent or nucleic acid array hybridization conditions to the RNA transcripts, or the complements thereof, of the corresponding rat genes. The probes can also be antibodies or other protein-binding molecules that can bind to the protein products of the corresponding rat genes with high affinities (e.g., at least 10⁶ M⁻¹, 10⁷ M⁻¹, 10⁸ M⁻¹, 10⁹ M⁻¹, or more). In one example, a probe array of the present invention includes one or more probes, each of which is directed to a rat gene that encodes a tiling sequence selected from WAN00OGR4, WAN00OGSD, WAN00OGSE, WAN00OGSH, WAN00OGSK, WAN00OGSN, WAN00OGSP, WAN00OGSQ, WAN00OGS4, and WAN00OGT4.

Various aspects of the invention are described in further detail in the following subsections. The use of subsections is not meant to limit the invention. Each subsection may apply to any aspect of the invention. In this application, the use of “or” means “and/or” unless otherwise stated.

A. Clustering of Rat Gene Sequences

mRNA, cDNA, and other coding or non-coding sequences of rat genes were collected from GenBank and other sources. The collected sequences were clustered and aligned using CAT (Clustering and Alignment Tool) software from DoubleTwist. See CLUSTERING AND ALIGNMENT TOOLS USER'S GUIDE (DoubleTwist, Inc., 2000). Each resulting cluster contained a set of highly homologous sequences that were aligned to derive consensus sequences. The consensus sequences were manually curated.

Examples of these consensus sequences are depicted in SEQ ID NOs: 1-3,519. Table 1 illustrates the headers for each consensus sequence. Each header includes a qualifier (e.g., “WAN00OE0” for SEQ ID NO: 1, “WAN00OE0R” for SEQ ID NO: 2, and so on) as well as other information of the corresponding rat gene.

The CAT program also generated exemplar sequences that did not cluster with any CAT sub-cluster. These exemplar sequences are depicted in SEQ ID NOs: 3,520-4,096. Table 2 provides the headers for these exemplar sequences.

The consensus and exemplar sequences are collectively referred to as the “parent sequences.” Any base ambiguity in a parent sequence is indicated according to the IUPAC (International Union of Pure and Applied Chemistry) guideline which is in consistence with WIPO Standard ST.25 (1998).

B. Preparation of Polynucleotide Probes for Rat Genes

The parent sequences depicted in SEQ ID NOs: 1-4,096 can be used to prepare polynucleotide probes for the corresponding rat genes. A polynucleotide probe for a rat gene can hybridize under stringent or nucleic acid array hybridization conditions to the RNA transcript(s), or the complement thereof, of the rat gene. Preferably, a polynucleotide probe for a rat gene is incapable of hybridizing under stringent or nucleic acid array hybridization conditions to the RNA transcripts, or the complements thereof, of other rat genes. In many embodiments, a polynucleotide probe for a rat gene can hybridize under stringent or nucleic acid array hybridization conditions to the parent sequence encoded by the gene, or the complement thereof, but not the parent sequences encoded by other rat genes or their complements.

Where a parent sequence contains an ambiguous residues (e.g., a “n” residue), the probes for the parent sequence can be designed to hybridize under stringent or nucleic acid array conditions to an unambiguous fragment of the parent sequence, or the complement of the unambiguous fragment. In one example, a probe for such a parent sequence comprises or consists of an unambiguous fragment of the parent sequence, or the complement thereof. In many instances, a polynucleotide probe for a parent sequence is incapable of hybridizing under stringent or nucleic acid array hybridization conditions to other parent sequences, or the complements thereof.

As used herein, “nucleic acid array hybridization conditions” refer to the temperature and ionic conditions that are normally employed in nucleic acid array hybridization. In many cases, the nucleic acid array hybridization conditions include 16-hour hybridization at 45° C., followed by at least three 10-minute washes at room temperature. The hybridization buffer includes 100 mM MES, 1 M [Na⁺], 20 mM EDTA, and 0.01% Tween 20. The wash buffer is 6×SSPET. 6×SSPET contains 0.9 M NaCl, 60 mM NaH₂PO₄, 6 mM EDTA, and 0.005% Triton X-100. Under more stringent nucleic acid array hybridization conditions, the wash buffer can be replaced with 100 mM MES, 0.1 M [Na⁺], and 0.01% Tween 20.

“Stringent conditions” are at least as stringent as, for example, conditions G-L in Table 3. In certain embodiments, highly stringent conditions A-F are employed. In Table 3, hybridization is carried out under the hybridization conditions (Hybridization Temperature and Buffer) for about four hours, followed by two 20-minute washes under the corresponding wash conditions (Wash Temp. and Buffer).

TABLE 3
Stringency Conditions

| Stringency | Poly-nucleotide | Hybrid | Hybridization | Wash Temp. |
|---|---|---|---|---|
| Condition | Hybrid | Length (bp)¹ | Temperature and Buffer^{H} | and Buffer^{H} |
| A | DNA:DNA | >50 | 65° C.; 1xSSC -or- | 65° C.; 0.3xSSC |
| | | | 42° C.; 1xSSC, 50% formamide | |
| B | DNA:DNA | <50 | T_{B}*; 1xSSC | T_{B}*; 1xSSC |
| C | DNA:RNA | >50 | 67° C.; 1xSSC -or- | 67° C.; 0.3xSSC |
| | | | 45° C.; 1xSSC, 50% formamide | |
| D | DNA:RNA | <50 | T_{D}*; 1xSSC | T_{D}*; 1xSSC |
| E | RNA:RNA | >50 | 70° C.; 1xSSC -or- | 70° C.; 0.3xSSC |
| | | | 50° C.; 1xSSC, 50% formamide | |
| F | RNA:RNA | <50 | T_{F}*; 1xSSC | T_{f}*; 1xSSC |
| G | DNA:DNA | >50 | 65° C.; 4xSSC -or- | 65° C.; 1xSSC |
| | | | 42° C.; 4xSSC, 50% formamide | |
| H | DNA:DNA | <50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC, 50% formamide | |
| J | DNA:RNA | <50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% formamide | |
| L | RNA:RNA | <50 | T_{L}*; 2xSSC | T_{L}*; 2xSSC |
| ¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| ^{H}SSPE (1xSSPE is 0.15M NaCl, 10 mM NaH₂PO₄, and 1.25 mM EDTA, pH 7.4) can be substituted for SSC (1xSSC is 0.15 M NaCl and 15 mM sodium citrate) in the hybridization and wash buffers. | | | | |
| T_{B}*-T_{R}*The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs in length, | | | | |
| # Tₘ(° C.) = 81.5 + 16.6(log₁₀Na⁺) + 0.41(% G + C) − (600/N), where N is the number of bases in the hybrid, and Na⁺ is the molar concentration of sodium ions in the hybridization buffer (Na⁺ for 1xSSC = 0.165 M). | | | | |

The length of a polynucleotide probe employed in the present invention can be selected to produce the desired hybridization effect. For example, a polynucleotide probe can be selected to have at least 10, 15, 20, 25, 30, 35, 40, 45, 50, 60, 70, 80, 90, 100, 200, 300, 400, or more nucleotide residues.

A polynucleotide probe of the present invention can include naturally occurring residues (e.g., deoxyadenylate, deoxycytidylate, deoxyguanylate, deoxythymidylate, adenylate, cytidylate, guanylate, or uridylate), synthetically-produced analogs, or combinations thereof. Examples of suitable synthetic analogs include, but are not limited to, aza or deaza pyrimidine analogs, aza or deaza purine analogs, and other heterocyclic base analogs, where one or more of the carbon and nitrogen atoms of the purine and pyrimidine rings are substituted by heteroatoms, such as oxygen, sulfur, selenium, and phosphorus.

The backbone of a polynucleotide probe of the present invention can employ a naturally occurring linkage (such as through 5′ to 3′ linkage), a modified linkage, or a combination thereof. In one embodiment, the nucleotide residues in a polynucleotide probe are covalently connected via a non-typical linkage, such as 5′ to 2′ linkage, provided that the linkage does not interfere with hybridization. In another embodiment, peptide nucleic acids, in which the constitute bases are joined by peptide bonds rather than phosphodiester linkages, are used.

In many cases, the polynucleotide probes of the present invention have relatively high sequence complexity and do not contain long stretches of the same nucleotide. In still many cases, each polynucleotide probe employed does not include any ambiguous residue. In one example, the polynucleotide probes of the present invention do not have a high proportion of G or C residues at the 3′ ends. In another example, the polynucleotide probes do not have a 3′ terminal T residue. Depending on the type of assay or detection to be performed, sequences that are predicted to form hairpins or interstrand structures, such as “primer dimers,” can be either included or excluded from the polynucleotide probes of the present invention.

Any part of a rat gene can be used to design polynucleotide probes. For instance, probes can be designed based on the protein-coding region, the 5′ untranslated region, or the 3′ untranslated region of a rat gene. Multiple probes, such as at least 5, 10, 15, 20, 25, 30, 40, 50, 60, 70, 80, 90, 100, or more probes, can be prepared for the same rat gene. These probes may or may not overlap each other, although overlap among probes is desirable in certain assays.

The polynucleotide probes for a parent sequence preferably have low sequence identity or similarity with other parent sequences or their complements. For instance, a polynucleotide probe for a parent sequence can have no more than 70%, 60%, 50%, or less sequence similarity with other parent sequences, or the complements thereof. This low sequence similarity reduces the risk of cross-hybridization. Sequence identity or similarity can be determined by a variety of algorithms, such as BLASTN, FASTA, FASTDB, or GCG programs.

The suitability of a polynucleotide probe for hybridization can be evaluated by numerous computer programs. Examples of these programs include, but are not limited to, LaserGene (DNAStar), Oligo (National Biosciences, Inc.), MacVector (Kodak/IBI), and GCG programs.

The polynucleotide probes of the present invention can be synthesized using any method known in the art. For instance, automated or high throughput DNA synthesizers can be employed to prepare polynucleotide probes. The synthesized probes can be purified by reverse phase chromatography, ethanol precipitation, gel filtration, electrophoresis, or other suitable means.

To facilitate the probe design, the parent sequences with relative large sizes can be divided into shorter sequence segments. These divided sequences, together with any undivided parent sequence, are collectively referred to as the “tiling sequences.” Examples of these tiling sequences are depicted in SEQ ID NOs: 4,097-8,192. Table 4 illustrates the headers for each tiling sequence. Each tiling sequence has the same qualifier as the corresponding parent sequence from which the tiling sequence is derived. Table 5 shows the location of each tiling sequence in the corresponding parent sequence. The 5′ and 3′ ends of each tiling sequence in the corresponding parent sequence are indicated under “TilingStart” and “TilingEnd,” respectively.

Table 4 includes the following tiling sequence: WAN00OGR4 (SEQ ID NO: 8084), WAN00OGRF (SEQ ID NO: 8085), WAN00OGS3 (SEQ ID NO: 8086), WAN00OGS4 (SEQ ID NO: 8087), WAN00OGS5 (SEQ ID NO: 8088), WAN00OGS6 (SEQ ID NO: 8089), WAN00OGS7 (SEQ ID NO: 8090), WAN00OGS8 (SEQ ID NO: 8091), WAN00OGS9 (SEQ ID NO: 8092), WAN00OGSA (SEQ ID NO: 8093), WAN00OGSB (SEQ ID NO: 8094), WAN00OGSC (SEQ ID NO: 7622), WAN00OGSD (SEQ ID NO: 8095), WAN00OGSE (SEQ ID NO: 8096), WAN00OGSF (SEQ ID NO: 8097), WAN00OGSG (SEQ ID NO: 8098), WAN00OGSH (SEQ ID NO: 8099), WAN00OGSI (SEQ ID NO: 8100), WAN00OGSJ (SEQ ID NO: 8101), WAN00OGSK (SEQ ID NO: 8102), WAN00OGSL (SEQ ID NO: 8103), WAN00OGSM (SEQ ID NO: 8104), WAN00OGSN (SEQ ID NO: 8105), WAN00OGSO (SEQ ID NO: 8106), WAN00OGSP (SEQ ID NO: 8107), WAN00OGSQ (SEQ ID NO: 8108), WAN00OGSR (SEQ ID NO: 8109), WAN00OGSS (SEQ ID NO: 8110), WAN00OGST (SEQ ID NO: 8111), WAN00OGSU (SEQ ID NO: 8112), WAN00OGSV (SEQ ID NO: 8113), WAN00OGSW (SEQ ID NO: 8114), WAN00OGSX (SEQ ID NO: 8115), WAN00OGSY (SEQ ID NO: 8116), WAN00OGSZ (SEQ ID NO: 8117), WAN00OGT0 (SEQ ID NO: 8118), WAN00OGT1 (SEQ ID NO: 8119), WAN00OGT2 (SEQ ID NO: 8120), WAN00OGT3 (SEQ ID NO: 8121), WAN00OGT4 (SEQ ID NO: 8122), WAN00OGT5 (SEQ ID NO: 8123), and WAN00OGT6 (SEQ ID NO: 8124).

A BLAST search of SEQ ID NO: 8084 (tiling:giRat1a:WAN00OGR4; WAN00OPIU CARD14) against the Rattus norvegicus genome database at National Center for Biotechnology Information (NCBI) did not produce any homologous sequence. SEQ ID NO: 8084 has over 98% sequence identity to human gene CARD14, which encodes member 14 of the caspase recruitment domain family. The CARD14 protein belongs to the membrane-associated guanylate kinase (MAGUK) family, a class of proteins that functions as molecular scaffolds for the assembly of multiprotein complexes at specialized regions of the plasma membrane. The protein is also a member of the CARD protein family, which is defined by carrying a characteristic caspase-associated recruitment domain (CARD). The CARD14 protein shares a similar domain structure with CARD11 protein. The CARD domains of both proteins have been shown to specifically interact with BCL10, a protein known to function as a positive regulator of cell apoptosis and NF-kappaB activation. When expressed in cells, the CARD14 protein can activate NF-kappaB and induce the phosphorylation of BCL10. At least two alternatively spliced isoforms have been reported for CARD14.

SEQ ID NO: 8085 (tiling:giRat1a:WAN00OGRF; WAN00OPIV zgrbrggam12xcpx) has about 100% sequence identify to rat gene LOC362381. LOC362381 is located on rat chromosome 4q31.

Nucleotides 1-1334 of SEQ ID NO: 8086 (tiling:giRat1a:WAN00OGS3; WAN00OPIW r84g5) have about 99% sequence identity to the rat gene that encodes histamine receptor H3. Histamine receptor H3 can inhibit forskolin-stimulated cAMP production in response to histamine. Histamine is a ubiquitous messenger molecule released from mast cells, enterochromaffin-like cells, and neurons. Its various actions are mediated by histamine receptors H1, H2, H3 and H4. Histamine receptor H3 belongs to the family 1 of G protein-coupled receptors. It is an integral membrane protein and can regulate neurotransmitter release. Histamine receptor H3 can also increase voltage-dependent calcium current in smooth muscles and innervates the blood vessels and the heart in cardiovascular system.

Fragments of SEQ ID NO: 8087 have sequence homology to a variety of rat genomic sequences. A BLAST search of SEQ ID NO: 8087 against the NCBI human genome did not produce any homologous sequence.

SEQ ID NO: 8088 (tiling:giRat1a:WAN00OGS5; WAN00OPIY NARC8) has at least 97% sequence identity to the rat gene that encodes nuclear receptor binding factor 1. Nucleotides 250-353 of SEQ ID NO: 8088 have about 90% sequence identity to human CGI-63, also known as NRBF1, which encodes nuclear receptor binding factor 1.

SEQ ID NO: 8089 (tiling:giRat1a:WAN00OGS6; WAN00OPIZ narc10a) has about 99% sequence identity to a rat genomic region on chromosome 4q24. The region is located within rat gene LOC362377 which encodes a protein similar to hect domain and RLD 3. Nucleotides 374 to 1343 of SEQ ID NO: 8089 have about 82% sequence identity to human gene NAP1L5 which encodes nucleosome assembly protein 1-like 5.

Nucleotides 1-1382 of SEQ ID NO: 8090 (tiling:giRat1a:WAN00OGS7; WAN00OPJ0 NARC13) have about 99% sequence identity to a rat genomic region on chromosome 19q12. The region is located between the protein-coding sequences of rat genes LOC292058 and LOC307913. LOC292058 encodes a protein similar to HSPC037 protein. LOC307913 encodes a protein similar to hypothetical protein KLAA0182. Nucleotides 1029 to 1375 of SEQ ID NO: 8090 have about 82% sequence identity to human gene KLAA0182.

SEQ ID NO: 8091 (tiling:giRat1a:WAN00OGS8; WAN00OPJ1 r8t) has about 98% sequence identity to rat gene Kcnip2. SEQ ID NO: 8091 aligns with the 3′ untranslated region and the protein-coding region of Kcnip2. Kcnip2 encodes Kv channel-interacting protein 2. The protein is a member of the family of voltage-gated potassium (Kv) channel-interacting proteins (KCNIPs), which belongs to the recoverin branch of the EF-hand superfamily. Members of the KCNIP family are small calcium binding proteins. Many members have EF-hand-like domains, and differ from each other in the N-terminus. They are integral subunit components of native Kv4 channel complexes. They may regulate A-type currents, and hence neuronal excitability, in response to changes in intracellular calcium. Multiple alternatively spliced transcript variants encoding distinct isoforms have been identified for this gene.

SEQ ID NO: 8092 (tiling:giRat1a:WAN00OGS9; WAN00OPJ2 rncs1) aligns with rat gene Hpca on chromosome 5q36. Hpca encodes hippocalcin, which is a member of neuron-specific calcium-binding proteins family found in the retina and brain. Hippocalcin is associated with the plasma membrane. It has similarities to proteins located in the photoreceptor cells that regulate photosignal transduction in a calcium-sensitive manner. Hippocalcin displays recoverin activity and a calcium-dependent inhibition of rhodopsin kinase.

SEQ ID NO: 8093 (tiling:giRat1a:WAN00OGSA; WAN00OPJ3 kchip2(9q)) has about 99% sequence identity to the 3′ untranslated region and the protein-coding region of rat gene Kcnip2.

SEQ ID NO: 8094 (tiling:giRat1a:WAN00OGSB; WAN00OPJ4 narc27) aligns with rat gene LOC298500. LOC298500 encodes a protein which is similar to hypothetical protein AL133206. LOC298500 is located on rat chromosome 5q36.

SEQ ID NO: 7622 (tiling:giRat1a:WAN00OGSC; WAN00OPJ5 r9q) aligns with a rat genomic region that overlaps the 3′ untranslated region of rat gene Kcnip2.

A BLAST search of SEQ ID NO: 8095 (tiling:giRat1a:WAN00OGSD; WAN00OPJ6 CARD6) against the NCBI rat genome produced no homologous sequence. SEQ ID NO: 8095 has about 99% sequence identity to a human genomic region that overlaps the 3′ untranslated region and the protein-coding region of human gene CARD6. CARD6 encodes member 6 of the caspase recruitment domain family. Members of the caspase recruitment domain family are defined by the presence of a characteristic caspase-associated recruitment domain (CARD). CARD is a protein interaction domain known to participate in activation or suppression of CARD containing members of the caspase family, and thus plays an important regulatory role in cell apoptosis.

A BLAST search of SEQ ID NO: 8096 (tiling:giRat1a:WAN00OGSE; WAN00OPJ7 CARD7) against the NCBI rat genome produced no homologous sequence. SEQ ID NO: 8096 has about 97% sequence identity to human gene NALP1. NALP1 encodes NACHT, leucine rich repeat and PYD containing 1, which is a member of the Ced4 family of apoptosis proteins. Ced-family members contain a caspase recruitment domain (CARD) and are known to be key mediators of programmed cell death. The NALP1 protein contains a distinct N-terminal pyrin-like motif, which is possibly involved in protein-protein interactions. This protein interacts strongly with caspase 2 and weakly with caspase 9. Overexpression of NALP1 gene can induce apoptosis in cells. Multiple alternatively spliced transcript variants encoding distinct isoforms have been found for NALP1 gene.

SEQ ID NO: 8097 (tiling:giRat1a:WAN00OGSF; WAN00OPJ8 zmrbr11pxcpx) aligns with rat gene LOC361071. LOC361071 encodes a protein similar to Succinyl-CoA ligase [ADP-forming] beta-chain, mitochondrial precursor (Succinyl-CoA synthetase, betaA chain) (SCS-betaA) (ATP-specific succinyl-CoA synthetase beta subunit). The gene is located on rat chromosome 15p11-q11. SEQ ID NO: 8097 has about 78-93% sequence identity to human gene SUCLA2 which encodes succinate-CoA ligase, ADP-forming, beta subunit.

SEQ ID NO: 8098 (tiling:giRat1a:WAN00OGSG; WAN00OPJ9 r1v) has about 98% sequence identity to rat gene Kcnip1. Kcnip1 encodes Kv channel interacting protein 1 which is a member of the family of voltage-gated potassium (Kv) channel-interacting proteins (KCNIPs).

A BLAST search of SEQ ID NO: 8099 (tiling:giRat1a:WAN00OGSH; WAN00OPJA CARD12) against the NCBI rat genome produced no homologous sequence. SEQ ID NO: 8099 has at least 99% sequence identity to human gene CARD12. CARD12 encodes member 12 of the caspase recruitment domain family.

Nucleotides 396-523 of SEQ ID NO: 8100 (tiling:giRat1a:WAN00OGSI; WAN00OPJB CARD9) have about 90% sequence identity to rat gene LOC360357. LOC360357 is a hypothetical gene supported by NM_{—}022303. SEQ ID NO: 8100 has about 96% sequence identity to human gene CARD9, which encodes member 9 of the caspase recruitment domain family.

A majority portion of SEQ ID NO: 8101 (tiling:giRat1a:WAN00OGSJ; WAN00OPJC narc16) aligns with the intron sequence of rat gene LOC362219. LOC362219 encodes hypothetical protein LK44.

A BLAST search of SEQ ID NO: 8102 (tiling:giRat1a:WAN00OGSK; WAN00OPJD CARD4) against the NCBO rat genome produced no homologous sequence. Nucleotides 1-1400 of SEQ ID NO: 8102 have about 99% sequence identity to human gene CARD4. CARD4 encodes member 4 of the caspase recruitment domain family.

Fragments of SEQ ID NO: 8103 (tiling:giRat1a:WAN00OGSL; WAN00OPJE narc6) have sequence homology to a variety of rat genomic sequences, including rat gene LOC363424. LOC363424 encodes a transcript similar to RIKEN cDNA 4933431D05.

Nucleotides 1-100 and 627-716 of SEQ ID NO: 8104 (tiling:giRat1a:WAN00OGSM; WAN00OPJF CARD3) have about 93-95% sequence identity to rat gene LOC362491. LOC362491 encodes a protein which is similar to receptor-interacting protein 2. SEQ ID NO: 8103 has about 99% sequence identity to human gene RIPK2, also known as CARD3, which encodes receptor-interacting serine-threonine kinase 2.

A BLAST search of SEQ ID NO: 8105 (tiling:giRat1a:WAN00OGSN; WAN00OPJG CARD10) against the NCBI rat genome produced no homologous sequence. Nucleotides 1-585 of SEQ ID NO: 8105 have about 99% sequence identity to human gene CARD10. CARD10 encodes member 10 of the caspase recruitment domain family.

Nucleotides 1-96 of SEQ ID NO: 8106 (tiling:giRat1a:WAN00OGSO; WAN00OPJH CARD11) have about 89% sequence identity to rat gene LOC304314. LOC304314 encodes a protein similar to member 11 of the caspase recruitment domain family. Nucleotides 1-585 of SEQ ID NO: 8106 have about 100% sequence identity to human gene CARD11, which encodes member 11 of the caspase recruitment domain family.

A BLAST search of SEQ ID NO: 8107 (tiling:giRat1a:WAN00OGSP; WAN00OPJI CARD5) against the NCBI rat genome produced no homologous sequence. Nucleotides 1-591 of SEQ ID NO: 8107 have 98% sequence identity to human gene ASC, also known as CARD5, which encodes apoptosis-associated speck-like protein containing a CARD. The ASC protein is an adaptor protein that is composed of two protein-protein interaction domains—namely an N-terminal PYRIN-PAAD-DAPIN domain (PYD) and a C-terminal caspase-recruitment domain (CARD). The PYD and CARD domains are members of the six-helix bundle death domain-fold superfamily that mediates assembly of large signaling complexes in the inflammatory and apoptotic signaling pathways via the activation of caspase. In normal cells, the ASC protein is localized to the cytoplasm. In cells undergoing apoptosis, the ASC protein forms ball-like aggregates near the nuclear periphery. At least three transcript variants encoding different isoforms have been reported for this gene.

A BLAST search of SEQ ID NO: 8108 (tiling:giRat1a:WAN00OGSQ; WAN00OPJJ CARD8) against the NCBI rat genome produced no homologous sequence. Nucleotides 1 to 600 of SEQ ID NO: 8108 have about 99% sequence identity to the 3′ untranslated region of human gene CARD8, which encodes member 8 of the caspase recruitment domain family.

SEQ ID NO: 8109 (tiling:giRat1a:WAN00OGSR; WAN00OPJK Caspase12) aligns with rat gene caspase 12. Increased expression of caspase 12 was observed in rat neurons after traumatic brain injury. The gene is believed to be involved in apoptosis.

SEQ ID NO: 8110 (tiling:giRat1a:WAN00OGSS; WAN00OPJL flr9o) has about 100% sequence identity to a rat genomic sequence located 3′ to the protein-coding region of LOC309828. LOC309828 encodes a transcript similar to RIKEN cDNA 2610102M01.

SEQ ID NO: 8111 (tiling:giRat1a:WAN00OGST; WAN00OPJM hkng) aligns with rat gene LOC367345. LOC367345 encodes a protein similar to clusterin-like 1 (retinal) (a prepropeptide specific to rod photoreceptor). The gene is located on rat chromosome 9q38.

SEQ ID NO: 8111 (tiling:giRat1a:WAN00OGSU; WAN00OPJN kv4.2(5′utr)) aligns with rat gene Kcnd2, which encodes potassium voltage gated channel, Shal-related family, member 2. The gene is located on rat chromosome 4q22.

A majority portion of SEQ ID NO: 8113 (tiling:giRat1a:WAN00OGSV; WAN00OPJ0 narc19) overlaps with the 3′ untranslated region of the rat gene that encodes epididymal secretory protein 1.

SEQ ID NO: 8114 (tiling:giRat1a:WAN00OGSW; WAN00OPJP narc9) has significant sequence identity to a rat genomic sequence located 3′ to the protein-coding region of rat gene LOC362219. LOC362219 is located on chromosome 3q36 and encodes hypothetical protein LK44.

SEQ ID NO: 8115 (tiling:giRat1a:WAN00OGSX; WAN00OPJQ PABLO) aligns with rat gene LOC294568, which encodes a protein similar to WASP family 1.

SEQ ID NO: 8116 (tiling:giRat1a:WAN00OGSY; WAN00OPJR r19r) aligns with rat gene Pitpn, which encodes phosphatidylinositol transfer protein. Phosphatidylinositol transfer protein is a member of cytosolic phospholipid transfer proteins.

SEQ ID NO: 8117 (tiling:giRat1a:WAN00OGSZ; WAN00OPJS rp19) aligns with rat gene Csen, which encodes calsenilin, presenilin binding protein, EF hand transcription factor. The Csen protein is a member of the family of voltage-gated potassium (Kv) channel-interacting proteins (KCNIPs), which belong to the recoverin branch of the EF-hand superfamily. The Csen protein can function as a calcium-regulated transcriptional repressor, and to interact with presenilins. Mutations in the presenilin genes have been implicated in Alzheimer's disease.

SEQ ID NO: 8118 (tiling:giRat1a:WAN00OGT0; WAN00OPJT zgrbrgbetalxcpx) has about 99% sequence identity to a rat genomic region which overlaps with the 3′ untranslated region of rat gene Gnb1. Gnb1 encodes guanine nucleotide binding protein, beta 1, and is located on chromosome 5q36. The Gnb1 protein is a component of heterotrimeric G-proteins. It can mediate activity of effector molecules and contribute to the specificity of G-protein receptor interaction.

SEQ ID NO: 8118 also has about 99% sequence identity to rat gene LOC301910, which encodes a protein similar to guanine nucleotide-binding protein, beta-1 subunit. LOC301910 is located on chromosome 1p11. Moreover, SEQ ID NO: 8118 has about 98% sequence identity to a rat genomic sequence locate on chromosome 16.

SEQ ID NO: 8119 (tiling:giRat1a:WAN00OGT1; WAN00OPJU zgrbrgbeta2xcpx) corresponds to a rat genomic sequence which is located 3′ to the protein-coding region of rat gene Gnb2. Gnb2 encodes guanine nucleotide binding protein, beta polypeptide 2. Many heterotrimeric guanine nucleotide-binding proteins (G proteins), which integrate signals between receptors and effector proteins, are composed of an alpha, a beta, and a gamma subunit. These subunits are encoded by families of related genes. The Gnb2 gene encodes a beta subunit. Beta subunits are important regulators of alpha subunits, as well as of certain signal transduction receptors and effectors.

SEQ ID NO: 8120 (tiling:giRat1a:WAN00OGT2; WAN00OPJV zgrbrgbeta4xcpx) has about 99% sequence identity to a genomic region on rat chromosome 2q25. The region is located between the protein-coding sequences of rat genes mitofusin 1 and LOC294962. LOC294962 encodes a protein similar to guanine nucleotide binding protein beta 4.

SEQ ID NO: 8121 (tiling:giRat1a:WAN00OGT3; WAN00OPJW zgrbrgbeta5xcpx) corresponds to rat gene Gnb5, which encodes guanine nucleotide binding protein beta 5.

A BLAST search of SEQ ID NO: 8122 (tiling:giRat1a:WAN00OGT4; WAN00OPJY zgrbrggam2xcpx) against the NCBI rat genome produced no homologous sequence.

SEQ ID NO: 8123 (tiling:giRat1a:WAN00OGT5; WAN00OPJZ zgrbrggam3xcpx) has significant sequence identity to a rat genomic region on chromosome 1q43. The region is located between the protein-coding sequences of rat genes LOC361722 and LOC361721. LOC361722 encodes a protein similar to seipin. LOC361721 encodes a protein similar to a hypothetical protein. SEQ ID NO: 8123 has about 96% sequence identity to human gene GNG3, which encodes guanine nucleotide binding protein, gamma 3.

SEQ ID NO: 8124 (tiling:giRat1a:WAN00OGT6; WAN00OPK1 PSGL-1) has significant sequence identity with rat gene LOC363930, which encodes a protein similar to P-selectin glycoprotein ligand precursor and is located on chromosome 12q16. Nucleotides 304-600 of SEQ ID NO: 8124 have about 81% sequence identity to human gene SELPLG, which encodes selectin P ligand. The SELPLG protein is the high affinity counter-receptor for P-selectin on myeloid cells and stimulated T lymphocytes. The protein may play a critical role in the tethering of these cells to activated platelets or endothelia expressing P-selectin. The organization of the human SELPG gene closely resembles that of human CD43 and the human platelet glycoprotein GpIb-alpha, both of which have an intron in the 5′ noncoding region, a long second exon containing the complete coding region, and TATA-less promoters.

Each tiling sequence depicted in Table 4 can be used to prepare polynucleotide probes for the corresponding rat gene(s). These polynucleotide probes can hybridize under stringent or nucleic acid array hybridization conditions to the tiling sequence, or the complement thereof. In one embodiment, a polynucleotide probe for a tiling sequence can hybridize under highly stringent conditions to the tiling sequence, or the complement thereof. In another embodiment, the polynucleotide probes for a tiling sequence are incapable of hybridizing under stringent or nucleic acid array hybridization conditions to other tiling sequences, or the complements thereof. Where a tiling sequence contains an ambiguous residue, the probes for the tiling sequence can be designed to hybridize under stringent or nucleic acid array conditions to an unambiguous segment of the tiling sequence, or the complement of the unambiguous segment.

In one embodiment, the polynucleotide probes for each tiling sequence are generated using Array Designer, a software package provided by TeleChem International, Inc (Sunnyvale, Calif. 94089). Examples of the probes thus generated are depicted in SEQ ID NOs: 8,193-174,863. The qualifiers of these probes are illustrated in Table 6. The qualifier of each probe is identical to that of the corresponding tiling sequence from which the probe is derived. Other methods or software programs can also be used to generate hybridization probes for the tiling sequences of the present invention.

The parent sequences, tiling sequences, and polynucleotide probes of the present invention can be used for expression profiling of rat genes. Methods suitable for this purpose include, but are not limited to, nucleic acid arrays (including bead arrays), Northern Blot, in situ hybridization, PCR, or RT-PCR.

C. Nucleic Acid Arrays for Expression Profiling of Rat Genes

The polynucleotide probes of the present invention can be used to make nucleic acid arrays. A typical nucleic acid array includes at least one substrate support which has a plurality of discrete regions. The location of each discrete region is either known or determinable. The discrete regions can be organized in various forms or patterns. In one example, the discrete regions are organized as an array of regularly spaced areas on a surface of the substrate support. Other regular or irregular patterns, such as linear, concentric or spiral patterns, can also be used.

Polynucleotide probes can be stably attached to the discrete regions via covalent or non-covalent interactions. By “stably attached” or “stably associated,” it means that a polynucleotide probe retains its position relative to the attached discrete region during nucleic acid array hybridization and subsequent signal detection. Any method known in the art can be used to stably attach a polynucleotide probe to a discrete region on a nucleic acid array. In one embodiment, the attachment is achieved by first depositing polynucleotide probes to the respective discrete regions and then exposing the substrate surface to a solution of a cross-linking agent, such as glutaraldehyde, borohydride, or other bifunctional agents. In another embodiment, polynucleotide probes are covalently bound to a substrate support via an alkylamino-linker group or by coating the glass slides with polyethylenimine followed by activation with cyanuric chloride for coupling the polynucleotides. In yet another embodiment, polynucleotide probes are covalently attached to a nucleic acid array through polymer linkers. The polymer linkers can improve the accessibility of the probes to their purported targets. The polymer linkers can be selected to minimize any interference with the interactions between the probes and the purported targets.

A polynucleotide probe can also be stably attached to a nucleic acid array via non-covalent interactions. In one embodiment, polynucleotide probes are attached to a substrate surface through electrostatic interactions between positively charged surface groups and the negatively charged probes. In another embodiment, the substrate support is a glass slide having a coating of a polycationic polymer on its surface, such as a cationic polypeptide, and polynucleotide probes are stably bound to these polycationic polymers. In still another embodiment, the methods described in U.S. Pat. No. 6,440,723 are used to stably attach polynucleotide probes to a nucleic acid array of the present invention.

Various materials can be used to make nucleic acid array substrate supports. Suitable materials include, but are not limited to, glass, silica, ceramics, nylons, quartz wafers, gels, metals, and papers. The substrate supports can be flexible or rigid. In one embodiment, the substrate supports are in the form of a tape which is wound up on a reel or cassette.

A nucleic acid array of the present invention can have only one substrate support. A nucleic acid array of the present invention can also include two or more substrate supports. In many cases, the substrate support(s) in a nucleic acid array is non-reactive with reagents that are used in nucleic acid array hybridization.

The surface(s) of a substrate support can be smooth and substantially planar. The surface(s) of a substrate support can also include a variety of configurations, such as raised or depressed regions, trenches, v-grooves, mesa structures, or other regular or irregular structures. The surface(s) of a substrate support can be coated with one or more modification layers. Suitable modification layers include inorganic or organic layers, such as metals, metal oxides, polymers, or small organic molecules. The surface(s) of a substrate support can also be chemically treated to include groups such as hydroxyl, carboxyl, amine, aldehyde, or sulfhydryl groups.

The discrete regions on a nucleic acid array can be of any size, shape, or density. For instance, they can be squares, ellipsoids, rectangles, triangles, or circles. Other regular or irregular geometric shapes can also be used. The discrete regions on a substrate support can have the same or different shapes. Each discrete region can have, for example, a surface area of less than 10⁻¹, 10⁻², 10⁻³, 10⁻⁴, 10⁻⁵, 10⁻⁶, 10⁻⁷ cm², cm², or lesser. The spacing between each discrete region and its closest neighbor, measured from center-to-center, can range, without limitation, from about 10 to about 400 μm. In a non-limiting example, the density of the discrete regions on a nucleic acid array is between 50 and 50,000 regions/cm².

Numerous methods can be used to make the nucleic acid arrays of the present invention. In one embodiment, polynucleotide probes are synthesized in a step-by-step manner on a substrate support. A variety of algorithms can be used to reduce the number of synthesis cycles. In one example, a nucleic acid array of the present invention is synthesized in a combinational fashion by delivering monomers to the discrete regions through mechanically constrained flowpaths. In another example, a nucleic acid array of the present invention is synthesized by spotting monomer reagents onto a substrate support using an ink jet printer, such as the DeskWriter C manufactured by Hewlett-Packard. In yet another example, photolithography techniques are used to immobilize polynucleotide probes. Polynucleotide probes can also be deposited to a substrate support in pre-synthesized forms.

The present invention features any type of nucleic acid array, such as oligonucleotide arrays, cDNA arrays, or bead arrays. A bead array includes a plurality of beads to which polynucleotide probes are stably attached.

A nucleic acid array of the present invention can include any number of polynucleotide probes. In many embodiments, a substantial portion of all probes on a nucleic acid array is probes for rat genes. For instance, rat gene probes can constitute at least 15%, 20%, 25%, 30%, 35%, 40%, 45%, or 50% of all polynucleotides (including perfect match and perfect mismatch probes) that are stably attached to a nucleic acid array of the present invention. These rat gene probes can be attached to one substrate support. They can also be attached to two or more substrate supports.

In one embodiment, a nucleic acid array of the present invention includes at least 2, 5, 10, 20, 30, 40, 50, 100, 200, 300, 400, 500, 1,000, 2,000, 3,000, 4,000, or more probes, each of which can hybridize under stringent or nucleic acid array hybridization conditions to a different respective rat gene. As used herein, a probe can hybridize to a gene if the probe can hybridize to an RNA transcript, or the complement thereof, of the gene.

In another embodiment, a nucleic acid array of the present invention includes at least 2, 5, 10, 20, 30, 40, 50, 100, 200, 300, 400, 500, 1,000, 2,000, 3,000, 4,000, or more probes, each of which can hybridize under stringent or nucleic acid array hybridization conditions to a different respective tiling sequence selected from SEQ ID NOs: 4,097-8,192, or the complement thereof. In yet another embodiment, a nucleic acid array of the present invention includes at least 2, 5, 10, 20, 30, 40, 50, 100, 200, 300, 400, 500, 1,000, 2,000, 3,000, 4,000, or more probes, each of which can hybridize under stringent or nucleic acid array hybridization conditions to a different respective rat gene that encodes a tiling sequence selected from SEQ ID NOs: 4,097-8,192. In a further embodiment, a nucleic acid array of the present invention comprises at least one probe for each tiling sequence selected from SEQ ID NOs: 4,097-8,192. As used herein, a probe for a sequence refers to a probe which can hybridize under stringent or nucleic acid hybridization conditions to the sequence or the complement thereof. In still yet another embodiment, a nucleic acid array of the present invention comprises at least one probe for each rat gene that encodes a tiling sequence selected from SEQ ID NOs: 4,097-8,192.

In another embodiment, a nucleic acid array of the present invention includes at least 2, 5, 10, 20, 30, 40, 50, 100, 200, 300, 400, 500, 1,000, 2,000, 3,000, 4,000, or more probes, each of which can hybridize under stringent or nucleic acid array hybridization conditions to a different respective parent sequence selected from SEQ ID NOs: 1-4,096, or the complement thereof. In yet another embodiment, a nucleic acid array of the present invention includes at least 2, 5, 10, 20, 30, 40, 50, 100, 200, 300, 400, 500, 1,000, 2,000, 3,000, 4,000, or more probes, each of which can hybridize under stringent or nucleic acid array hybridization conditions to a different respective rat gene that encodes a parent sequence selected from SEQ ID NOs: 1-4,096.

In a further embodiment, a nucleic acid array of the present invention includes at least 1, 2, 3, 4, 5, 6, 7, 8, 9, 10, 15, 20, 25, 30, 35, 40, or 42 polynucleotide probes, each of which can hybridize under stringent or nucleic acid array hybridization conditions to a different respective rat gene that encodes a sequence selected from the group consisting of WAN00OGR4 (SEQ ID NO: 8084), WAN00OGRF (SEQ ID NO: 8085), WAN00OGS3 (SEQ ID NO: 8086), WAN00OGS4 (SEQ ID NO: 8087), WAN00OGS5 (SEQ ID NO: 8088), WAN00OGS6 (SEQ ID NO: 8089), WAN00OGS7 (SEQ ID NO: 8090), WAN00OGS8 (SEQ ID NO: 8091), WAN00OGS9 (SEQ ID NO: 8092), WAN00OGSA (SEQ ID NO: 8093), WAN00OGSB (SEQ ID NO: 8094), WAN00OGSC (SEQ ID NO: 7622), WAN00OGSD (SEQ ID NO: 8095), WAN00OGSE (SEQ ID NO: 8096), WAN00OGSF (SEQ ID NO: 8097), WAN00OGSG (SEQ ID NO: 8098), WAN00OGSH (SEQ ID NO: 8099), WAN00OGSI (SEQ ID NO: 8100), WAN00OGSJ (SEQ ID NO: 8101), WAN00OGSK (SEQ ID NO: 8102), WAN00OGSL (SEQ ID NO: 8103), WAN00OGSM (SEQ ID NO: 8104), WAN00OGSN (SEQ ID NO: 8105), WAN00OGSO (SEQ ID NO: 8106), WAN00OGSP (SEQ ID NO: 8107), WAN00OGSQ (SEQ ID NO: 8108), WAN00OGSR (SEQ ID NO: 8109), WAN00OGSS (SEQ ID NO: 8110), WAN00OGST (SEQ ID NO: 8111), WAN00OGSU (SEQ ID NO: 8112), WAN00OGSV (SEQ ID NO: 8113), WAN00OGSW (SEQ ID NO: 8114), WAN00OGSX (SEQ ID NO: 8115), WAN00OGSY (SEQ ID NO: 8116), WAN00OGSZ (SEQ ID NO: 8117), WAN00OGT0 (SEQ ID NO: 8118), WAN00OGT1 (SEQ ID NO: 8119), WAN00OGT2 (SEQ ID NO: 8120), WAN00OGT3 (SEQ ID NO: 8121), WAN00OGT4 (SEQ ID NO: 8122), WAN00OGT5 (SEQ ID NO: 8123), and WAN00OGT6 (SEQ ID NO: 8124).

In another embodiment, a nucleic acid array of the present invention includes at least 1, 2, 3, 4, 5, 6, 7, 8, 9, or 10 probes, each of which can hybridize under stringent or nucleic acid array hybridization conditions to a different respective rat gene that encodes a sequence selected from the group consisting of WAN00OGR4 (SEQ ID NO: 8084), WAN00OGSD (SEQ ID NO: 8095), WAN00OGSE (SEQ ID NO: 8096), WAN00OGSH (SEQ ID NO: 8099), WAN00OGSK (SEQ ID NO: 8102), WAN00OGSN (SEQ ID NO: 8105), WAN00OGSP (SEQ ID NO: 8107), WAN00OGSQ (SEQ ID NO: 8108), WAN00OGS4 (SEQ ID NO: 8087), and WAN00OGT4 (SEQ ID NO: 8122).

In still another embodiment, a nucleic acid array of the present invention includes at least one probe which can hybridize under stringent or nucleic acid array hybridization conditions to a rat gene that encodes WAN00OGS4 (SEQ ID NO: 8087), and/or at least one probe which can hybridize under stringent or nucleic acid array hybridization conditions to a rat gene that encodes WAN00OGT4 (SEQ ID NO: 8122).

Multiple probes for the same rat gene or tiling sequence can be included in a nucleic acid array of the present invention. For instance, at least 2, 5, 10, 15, 20, 25, 30, 35, 40, 45, 50, 55, 60, 65, 70, or more probes can be used for each rat gene or tiling sequence being analyzed. In many instances, reliability and reproducibility of the probe set signal values decrease substantially if less than 20 probe pairs per transcript are used. By increasing the number of probe pairs for each rat gene or tile sequence, a more robust and reliable detection can be produced.

Different polynucleotide probes can be attached to different respective discrete regions on a nucleic acid array of the present invention. Different probes can also be attached to the same discrete region. The concentration of one probe with respect to other probe or probes in the same region may vary according to the objectives and requirements of the particular experiment. In one example, different probes in the same region are present in approximately equimolar ratio. Likewise, probes for different rat genes or tiling sequences can be attached to the same or different discrete regions on a nucleic acid array.

A nucleic acid array of the present invention can further include control probes which can hybridize under stringent or nucleic acid array hybridization conditions to respective control sequences, or the complements thereof. Examples of suitable control sequences are depicted in SEQ ID NO: 174,864-174,982. Table 7 illustrates the headers for each control sequence. Each header includes a qualifier as well as other information of the corresponding control sequence.

TABLE 7
Control Sequences

| SEQ ID | Header |
|---|---|
| 174864 | >control: giRat1a: Unassigned; Rat 18S rRNA gene, complete. |
| 174865 | >control: giRat1a: Unassigned; Rat 18S rRNA gene, complete. |
| 174866 | >control: giRat1a: Unassigned; Rat 18S rRNA gene, complete. |
| 174867 | >control: giRat1a: Unassigned; R. norvegicus 5S rRNA gene (clone pRA5S2). |
| 174868 | >control: giRat1a: Unassigned; Rat gene encoding cytoplasmic beta-actin. |
| 174869 | >control: giRat1a: Unassigned; Rat gene encoding cytoplasmic beta-actin. |
| 174870 | >control: giRat1a: Unassigned; Rat gene encoding cytoplasmic beta-actin. |
| 174871 | >control: giRat1a: Unassigned; Rat mRNA for glyceraldehyde-3-phosphate- |
| | dehydrogenase (GAPDH) (GAPDH, EC 1.2.1.12). |
| 174872 | >control: giRat1a: Unassigned; Rat mRNA for glyceraldehyde-3-phosphate- |
| | dehydrogenase (GAPDH) (GAPDH, EC 1.2.1.12). |
| 174873 | >control: giRat1a: Unassigned; Rat mRNA for glyceraldehyde-3-phosphate- |
| | dehydrogenase (GAPDH) (GAPDH, EC 1.2.1.12). |
| 174874 | >control: giRat1a: Unassigned; Rattus norvegicus brain hexokinase mRNA, |
| | complete cds. |
| 174875 | >control: giRat1a: Unassigned; Rattus norvegicus brain hexokinase mRNA, |
| | complete cds. |
| 174876 | >control: giRat1a: Unassigned; Rattus norvegicus brain hexokinase mRNA, |
| | complete cds. |
| 174877 | >control: giRat1a: Unassigned; Rat DNA for B1 repeat (1-42) from gamma |
| | crystallin gene cluster. |
| 174878 | >control: giRat1a: Unassigned; Rat DNA for B2 repeat (1-12) from gamma |
| | crystallin gene cluster. |
| 174879 | >control: giRat1a: J04423; J04423 E coli bioB gene biotin synthetase (-5, -M, -3 |
| | represent transcript regions 5 prime, Middle, and 3 prime respectively) |
| 174880 | >control: giRat1a: J04423; J04423 E coli bioB gene biotin synthetase (-5, -M, -3 |
| | represent transcript regions 5 prime, Middle, and 3 prime respectively) |
| 174881 | >control: giRat1a: J04423; J04423 E coli bioB gene biotin synthetase (-5, -M, -3 |
| | represent transcript regions 5 prime, Middle, and 3 prime respectively) |
| 174882 | >control: giRat1a: J04423; J04423 E coli bioC protein (-5 and -3 represent |
| | transcript regions 5 prime and 3 prime respectively) |
| 174883 | >control: giRat1a: J04423; J04423 E coli bioC protein (-5 and -3 represent |
| | transcript regions 5 prime and 3 prime respectively) |
| 174884 | >control: giRat1a: J04423; J04423 E coli bioD gene dethiobiotin synthetase (-5 |
| | and -3 represent transcript regions 5 prime and 3 prime respectively) |
| 174885 | >control: giRat1a: J04423; J04423 E coli bioD gene dethiobiotin synthetase (-5 |
| | and -3 represent transcript regions 5 prime and 3 prime respectively) |
| 174886 | >control: giRat1a: X03453; X03453 Bacteriophage P1 cre recombinase protein (-5 |
| | and -3 represent transcript regions 5 prime and 3 prime respectively) |
| 174887 | >control: giRat1a: X03453; X03453 Bacteriophage P1 cre recombinase protein (-5 |
| | and -3 represent transcript regions 5 prime and 3 prime respectively) |
| 174888 | >control: giRat1a: L38424; L38424 B subtilis dapB, jojF, jojG genes |
| | corresponding to nucleotides 1358-3197 of L38424 (-5, -M, -3 represent |
| | transcript regions 5 prime, Middle, and 3 prime respectively) |
| 174889 | >control: giRat1a: L38424; L38424 B subtilis dapB, jojF, jojG genes |
| | corresponding to nucleotides 1358-3197 of L38424 (-5, -M, -3 represent |
| | transcript regions 5 prime, Middle, and 3 prime respectively) |
| 174890 | >control: giRat1a: L38424; L38424 B subtilis dapB, jojF, jojG genes |
| | corresponding to nucleotides 1358-3197 of L38424 (-5, -M, -3 represent |
| | transcript regions 5 prime, Middle, and 3 prime respectively) |
| 174891 | >control: giRat1a: X17013; X17013 B subtilis lys gene for diaminopimelate |
| | decarboxylase corresponding to nucleotides 350-1345 of X17013 (-5, -M, -3 |
| | represent transcript regions 5 prime, Middle, and 3 prime respectively) |
| 174892 | >control: giRat1a: X17013; X17013 B subtilis lys gene for diaminopimelate |
| | decarboxylase corresponding to nucleotides 350-1345 of X17013 (-5, -M, -3 |
| | represent transcript regions 5 prime, Middle, and 3 prime respectively) |
| 174893 | >control: giRat1a: X17013; X17013 B subtilis lys gene for diaminopimelate |
| | decarboxylase corresponding to nucleotides 350-1345 of X17013 (-5, -M, -3 |
| | represent transcript regions 5 prime, Middle, and 3 prime respectively) |
| 174894 | >control: giRat1a: M24537; M24537 B subtilis pheB, pheA genes corresponding |
| | to nucleotides 2017-3334 of M24537 (-5, -M, -3 represent transcript regions 5 |
| | prime, Middle, and 3 prime respectively) |
| 174895 | >control: giRat1a: M24537; M24537 B subtilis pheB, pheA genes corresponding |
| | to nucleotides 2017-3334 of M24537 (-5, -M, -3 represent transcript regions 5 |
| | prime, Middle, and 3 prime respectively) |
| 174896 | >control: giRat1a: M24537; M24537 B subtilis pheB, pheA genes corresponding |
| | to nucleotides 2017-3334 of M24537 (-5, -M, -3 represent transcript regions 5 |
| | prime, Middle, and 3 prime respectively) |
| 174897 | >control: giRat1a: X04603; X04603 B subtilis thrC, thrB genes corresponding to |
| | nucleotides 248-2229 of X04603 (-5, -M, -3 represent transcript regions 5 prime, |
| | Middle, and 3 prime respectively) |
| 174898 | >control: giRat1a: X04603; X04603 B subtilis thrC, thrB genes corresponding to |
| | nucleotides 248-2229 of X04603 (-5, -M, -3 represent transcript regions 5 prime, |
| | Middle, and 3 prime respectively) |
| 174899 | >control: giRat1a: K01391; K01391 B subtilis TrpE protein, TrpD protein, TrpC |
| | protein corresponding to nucleotides 1883-4400 of K01391 (-5, -M, -3 represent |
| | transcript regions 5 prime, Middle, and 3 prime respectively) |
| 174900 | >control: giRat1a: K01391; K01391 B subtilis TrpE protein, TrpD protein, TrpC |
| | protein corresponding to nucleotides 1883-4400 of K01391 (-5, -M, -3 represent |
| | transcript regions 5 prime, Middle, and 3 prime respectively) |
| 174901 | >control: giRat1a: K01391; K01391 B subtilis TrpE protein, TrpD protein, TrpC |
| | protein corresponding to nucleotides 1883-4400 of K01391 (-5, -M, -3 represent |
| | transcript regions 5 prime, Middle, and 3 prime respectively) |
| 174902 | >control: giRat1a: Unassigned; Rat gene encoding cytoplasmic beta-actin. |
| 174903 | >control: giRat1a: Unassigned; Rat gene encoding cytoplasmic beta-actin. |
| 174904 | >control: giRat1a: Unassigned; Rat gene encoding cytoplasmic beta-actin. |
| 174905 | >control: giRat1a: Unassigned; E. coli biotin synthetase (bioB), complete cds. |
| 174906 | >control: giRat1a: Unassigned; E. coli biotin synthetase (bioB), complete cds. |
| 174907 | >control: giRat1a: Unassigned; E. coli biotin synthetase (bioB), complete cds. |
| 174908 | >control: giRat1a: Unassigned; E. coli biotin synthetase (bioB), complete cds. |
| 174909 | >control: giRat1a: Unassigned; E. coli biotin synthetase (bioB), complete cds. |
| 174910 | >control: gikat1a: Unassigned; E. coli biotin synthetase (bioB), complete cds. |
| 174911 | >control: giRat1a: Unassigned; E. coli biotin synthetase (bioB), complete cds. |
| 174912 | >control: giRat1a: Unassigned; E. coli biotin synthetase (bioB), complete cds. |
| 174913 | >control: giRat1a: Unassigned; E. coli biotin synthetase (bioB), complete cds. |
| 174914 | >control: giRat1a: Unassigned; E. coli biotin synthetase (bioB), complete cds. |
| 174915 | >control: giRat1a: Unassigned; E. coli biotin synthetase (bioB), complete cds. |
| 174916 | >control: giRat1a: Unassigned; E. coli biotin synthetase (bioB), complete cds. |
| 174917 | >control: giRat1a: Unassigned; E. coli biotin synthetase (bioB), complete cds. |
| 174918 | >control: giRat1a: Unassigned; E. coli biotin synthetase (bioB), complete cds. |
| 174919 | >control: giRat1a: Unassigned; E. coli biotin synthetase (bioB), complete cds. |
| 174920 | >control: giRat1a: Unassigned; E. coli bioC protein, complete cds. |
| 174921 | >control: giRat1a: Unassigned; E. coli bioC protein, complete cds. |
| 174922 | >control: giRat1a: Unassigned; E. coli bioC protein, complete cds. |
| 174923 | >control: giRat1a: Unassigned; E. coli bioC protein, complete cds. |
| 174924 | >control: giRat1a: Unassigned; E. coli bioC protein, complete cds. |
| 174925 | >control: giRat1a: Unassigned; E. coli bioC protein, complete cds. |
| 174926 | >control: giRat1a: Unassigned; E. coli bioC protein, complete cds. |
| 174927 | >control: giRat1a: Unassigned; E. coli bioC protein, complete cds. |
| 174928 | >control: giRat1a: Unassigned; E. coli bioC protein, complete cds. |
| 174929 | >control: giRat1a: Unassigned; E. coli bioC protein, complete cds. |
| 174930 | >control: giRat1a: Unassigned; E. coli dethiobiotin synthetase (bioD), complete |
| | cds. |
| 174931 | >control: giRat1a: Unassigned; E. coli dethiobiotin synthetase (bioD), complete |
| | cds. |
| 174932 | >control: giRat1a: Unassigned; E. coli dethiobiotin synthetase (bioD), complete |
| | cds. |
| 174933 | >control: giRat1a: Unassigned; E. coli dethiobiotin synthetase (bioD), complete |
| | cds. |
| 174934 | >control: giRat1a: Unassigned; E. coli dethiobiotin synthetase (bioD), complete |
| | cds. |
| 174935 | >control: giRat1a: Unassigned; E. coli dethiobiotin synthetase (bioD), complete |
| | cds. |
| 174936 | >control: giRat1a: Unassigned; Bacteriophage P1 cre gene for recombinase |
| | protein. |
| 174937 | >control: giRat1a: Unassigned; Bacteriophage P1 cre gene for recombinase |
| | protein. |
| 174938 | >control: giRat1a: Unassigned; Bacteriophage P1 cre gene for recombinase |
| | protein. |
| 174939 | >control: giRat1a: Unassigned; Bacteriophage P1 cre gene for recombinase |
| | protein. |
| 174940 | >control: giRat1a: Unassigned; Bacteriophage P1 cre gene for recombinase |
| | protein. |
| 174941 | >control: giRat1a: Unassigned; Bacteriophage P1 cre gene for recombinase |
| | protein. |
| 174942 | >control: giRat1a: Unassigned; Bacteriophage P1 cre gene for recombinase |
| | protein. |
| 174943 | >control: giRat1a: Unassigned; Bacteriophage P1 cre gene for recombinase |
| | protein. |
| 174944 | >control: giRat1a: Unassigned; Bacteriophage P1 cre gene for recombinase |
| | protein. |
| 174945 | >control: giRat1a: Unassigned; Bacteriophage P1 cre gene for recombinase |
| | protein. |
| 174946 | >control: giRat1a: Unassigned; Bacillus subtilis dihydropicolinate reductase |
| | (dapB), jojF, jojG, complete cds's. |
| 174947 | >control: giRat1a: Unassigned; Bacillus subtilis dihydropicolinate reductase |
| | (dapB), jojF, jojG, complete cds's. |
| 174948 | >control: giRat1a: Unassigned; Bacillus subtilis dihydropicolinate reductase |
| | (dapB), jojF, jojG, complete cds's. |
| 174949 | >control: giRat1a: Unassigned; Bacillus subtilis dihydropicolinate reductase |
| | (dapB), jojF, jojG, complete cds's. |
| 174950 | >control: giRat1a: Unassigned; Bacillus subtilis dihydropicolinate reductase |
| | (dapB), jojF, jojG, complete cds's. |
| 174951 | >control: giRat1a: Unassigned; Bacillus subtilis dihydropicolinate reductase |
| | (dapB), jojF, jojG, complete cds's. |
| 174952 | >control: giRat1a: Unassigned; Bacillus subtilis dihydropicolinate reductase |
| | (dapB), jojF, jojG, complete cds's. |
| 174953 | >control: giRat1a: Unassigned; Bacillus subtilis dihydropicolinate reductase |
| | (dapB), jojF, jojG, complete cds's. |
| 174954 | >control: giRat1a: Unassigned; Bacillus subtilis dihydropicolinate reductase |
| | (dapB), jojF, jojG, complete cds's. |
| 174955 | >control: giRat1a: Unassigned; Bacillus subtilis dihydropicolinate reductase |
| | (dapB), jojF, jojG, complete cds's. |
| 174956 | >control: giRat1a: Unassigned; Bacillus subtilis dihydropicolinate reductase |
| | (dapB), jojF, jojG, complete cds's. |
| 174957 | >control: giRat1a: Unassigned; Bacillus subtilis dihydropicolinate reductase |
| | (dapB), jojF, jojG, complete cds's. |
| 174958 | >control: giRat1a: Unassigned; Bacillus subtilis dihydropicolinate reductase |
| | (dapB), jojF, jojG, complete cds's. |
| 174959 | >control: giRat1a: Unassigned; Bacillus subtilis dihydropicolinate reductase |
| | (dapB), jojF, jojG, complete cds's. |
| 174960 | >control: giRat1a: Unassigned; Bacillus subtilis dihydropicolinate reductase |
| | (dapB), jojF, jojG, complete cds's. |
| 174961 | >control: giRat1a: Unassigned; Rat glyceraldehyde-3-phosphate-dehydrogenase |
| | (GAPDH) mRNA, complete cds. |
| 174962 | >control: giRat1a: Unassigned; Rat glyceraldehyde-3-phosphate-dehydrogenase |
| | (GAPDH) mRNA, complete cds. |
| 174963 | >control: giRat1a: Unassigned; Rat glyceraldehyde-3-phosphate-dehydrogenase |
| | (GAPDH) mRNA, complete cds. |
| 174964 | >control: giRat1a: Unassigned; Bacillus subtilis lys gene for diaminopimelate |
| | decarboxylase (EC 4.1.1.20). |
| 174965 | >control: giRat1a: Unassigned; Bacillus subtilis lys gene for diaminopimelate |
| | decarboxylase (EC 4.1.1.20). |
| 174966 | >control: giRat1a: Unassigned; Bacillus subtilis lys gene for diaminopimelate |
| | decarboxylase (EC 4.1.1.20). |
| 174967 | >control: giRat1a: Unassigned; Bacillus subtillis phenylalanine biosynthesis |
| | associated protein (pheB), and monofunctional prephenate dehydratase (pheA) |
| | genes, complete cds. |
| 174968 | >control: giRat1a: Unassigned; Bacillus subtillis phenylalanine biosynthesis |
| | associated protein (pheB), and monofunctional prephenate dehydratase (pheA) |
| | genes, complete cds. |
| 174969 | >control: giRat1a: Unassigned; Bacillus subtillis phenylalanine biosynthesis |
| | associated protein (pheB), and monofunctional prephenate dehydratase (pheA) |
| | genes, complete cds. |
| 174970 | >control: giRat1a: Unassigned; Rattus norvegicus pyruvate carboxylase mRNA, |
| | complete cds. |
| 174971 | >control: giRat1a: Unassigned; Rattus norvegicus pyruvate carboxylase mRNA, |
| | complete cds. |
| 174972 | >control: giRat1a: Unassigned; Rattus norvegicus pyruvate carboxylase mRNA, |
| | complete cds. |
| 174973 | >control: giRat1a: Unassigned; Rattus norvegicus pyruvate carboxylase mRNA, |
| | complete cds. |
| 174974 | >control: giRat1a: Unassigned; B. subtilis thrB and thrC genes for homoserine |
| | kinase and threonine synthase (EC 2.7.1.39 and EC 4.2.99.2, respectively). |
| 174975 | >control: giRat1a: Unassigned; B. subtilis thrB and thrC genes for homoserine |
| | kinase and threonine synthase (EC 2.7.1.39 and EC 4.2.99.2, respectively). |
| 174976 | >control: giRat1a: Unassigned; B. subtilis thrB and thrC genes for homoserine |
| | kinase and threonine synthase (EC 2.7.1.39 and EC 4.2.99.2, respectively). |
| 174977 | >control: giRat1a: Unassigned; Rat transferrin receptor mRNA, 3′ end. |
| 174978 | >control: giRat1a: Unassigned; Rat transferrin receptor mRNA, 3′ end. |
| 174979 | >control: giRat1a: Unassigned; Rat transferrin receptor mRNA, 3′ end. |
| 174980 | >control: giRat1a: Unassigned; B. subtilis tryptophan (trp) operon, complete cds. |
| 174981 | >control: giRat1a: Unassigned; B. subtilis tryptophan (trp) operon, complete cds. |
| 174982 | >control: giRat1a: Unassigned; B. subtilis tryptophan (trp) operon, complete cds. |

In many embodiments, a nucleic acid array of the present invention also includes mismatch probes for each perfect match probe. Suitable mismatch probes include, without limitation, perfect mismatch probes. A perfect mismatch probe has the same sequence as the corresponding perfect match probe except for a homomeric substitution (A to T, T to A, G to C, and C to G) at or near the center of the mismatch probe. For instance, if a perfect match probe has 2n nucleotides, the homomeric substitution in the corresponding perfect mismatch probe is either at the n or n+1 position, but not at both positions. If a perfect match probe has 2n+1 nucleotides, the homomeric substitution in the corresponding perfect mismatch probe is at the n+1 position. The center location of the mismatched residue is more likely to destabilize the duplex formed with the target sequence under the hybridization conditions. Each perfect match probe and the corresponding perfect mistmatch are typically attached to different regions on a nucleic acid array.

In one example, a nucleic acid array of the present invention includes each and every polynucleotide probe selected from SEQ ID NOs: 8,193-174,863 (or the complement thereof). In another example, a nucleic acid array of the present invention includes each and every polynucleotide probe selected from SEQ ID NOs: 8,193-174,863 (or the complement thereof), and its perfect mismatch probe.

D. Protein Arrays for Expression Profiling of Rat Genes

The present invention also features protein arrays for the detection, quantitation, and differential expression analysis of rat genes. Each protein array of the present invention includes probes that can specifically bind to the protein products of corresponding rat genes. In one embodiment, the probes on a protein array of the present invention are antibodies, and each antibody can bind to the corresponding rat protein with an affinity constant of at least 10⁴ M⁻¹, 10⁵ M⁻¹, 10⁶ M⁻¹, 10⁷ M⁻¹, 10⁸ M⁻¹, 10⁹ M⁻¹, or more. In many instances, the antibody does not bind to other rat proteins. Antibodies suitable for this purpose include, but are not limited to, polyclonal antibodies, monoclonal antibodies, chimeric antibodies, single chain antibodies, Fab fragments, or fragments produced by Fab expression libraries. Other peptides, scaffolds, or protein-binding molecules can also be used to construct the protein arrays of the present invention.

Antibodies or other protein-binding molecules can be immobilized to a protein array using a variety of methods. Examples of these methods include, but are not limited to, diffusion (e.g., agarose or polyacrylamide gel), surface absorption (e.g., nitrocellulose or PVDF), covalent binding (e.g., silanes or aldehyde), or non-covalent affinity binding (e.g., biotin-streptavidin). Examples of protein array fabrication methods include, but are not limited to, ink-jetting, robotic contact printing, photolithography, or piezoelectric spotting. The method described in MacBeath and Schreiber, SCIENCE, 289: 1760-1763 (2000) can also be used. Suitable substrate supports for a protein array of the present invention include, but are not limited to, glass, membranes, mass spectrometer plates, microtiter wells, silica, or beads.

In one embodiment, a protein array of the present invention includes at least 2, 5, 10, 20, 30, 40, 50, 100, 200, 300, 400, 500, 1,000, 2,000, 3,000, 4,000, or more probes, and each of these probes can specifically bind to a different respective rat protein. In another embodiment, a protein array of the present invention includes at least 2, 5, 10, 20, 30, 40, 50, 100, 200, 300, 400, 500, 1,000, 2,000, 3,000, 4,000, or more probes, and each of these probes can bind to a protein product of a different respective rat gene that encodes a sequence selected from SEQ ID NOs: 4,097-8,192.

In yet another embodiment, a protein array of the present invention includes at least 1, 2, 3, 4, 5, 6, 7, 8, 9, 10, 20, 30, 40, or 42 probes, and each of these probes can specifically bind to a protein product of a different respective rat gene that encodes a sequence selected from the group consisting of WAN00OGR4 (SEQ ID NO: 8084), WAN00OGRF (SEQ ID NO: 8085), WAN00OGS3 (SEQ ID NO: 8086), WAN00OGS4 (SEQ ID NO: 8087), WAN00OGS5 (SEQ ID NO: 8088), WAN00OGS6 (SEQ ID NO: 8089), WAN00OGS7 (SEQ ID NO: 8090), WAN00OGS8 (SEQ ID NO: 8091), WAN00OGS9 (SEQ ID NO: 8092), WAN00OGSA (SEQ ID NO: 8093), WAN00OGSB (SEQ ID NO: 8094), WAN00OGSC (SEQ ID NO: 7622), WAN00OGSD (SEQ ID NO: 8095), WAN00OGSE (SEQ ID NO: 8096), WAN00OGSF (SEQ ID NO: 8097), WAN00OGSG (SEQ ID NO: 8098), WAN00OGSH (SEQ ID NO: 8099), WAN00OGSI (SEQ ID NO: 8100), WAN00OGSJ (SEQ ID NO: 8101), WAN00OGSK (SEQ ID NO: 8102), WAN00OGSL (SEQ ID NO: 8103), WAN00OGSM (SEQ ID NO: 8104), WAN00OGSN (SEQ ID NO: 8105), WAN00OGSO (SEQ ID NO: 8106), WAN00OGSP (SEQ ID NO: 8107), WAN00OGSQ (SEQ ID NO: 8108), WAN00OGSR (SEQ ID NO: 8109), WAN00OGSS (SEQ ID NO: 8110), WAN00OGST (SEQ ID NO: 8111), WAN00OGSU (SEQ ID NO: 8112), WAN00OGSV (SEQ ID NO: 8113), WAN00OGSW (SEQ ID NO: 8114), WAN00OGSX (SEQ ID NO: 8115), WAN00OGSY (SEQ ID NO: 8116), WAN00OGSZ (SEQ ID NO: 8117), WAN00OGT0 (SEQ ID NO: 8118), WAN00OGT1 (SEQ ID NO: 8119), WAN00OGT2 (SEQ ID NO: 8120), WAN00OGT3 (SEQ ID NO: 8121), WAN00OGT4 (SEQ ID NO: 8122), WAN00OGT5 (SEQ ID NO: 8123), and WAN00OGT6 (SEQ ID NO: 8124).

In still another embodiment, a protein array of the present invention includes at least 1, 2, 3, 4, 5, 6, 7, 8, 9, or 10 probes, each of which can specifically bind to a protein product of a different respective rat gene that encodes a sequence selected from the group consisting of WAN00OGR4 (SEQ ID NO: 8084), WAN00OGSD (SEQ ID NO: 8095), WAN00OGSE (SEQ ID NO: 8096), WAN00OGSH (SEQ ID NO: 8099), WAN00OGSK (SEQ ID NO: 8102), WAN00OGSN (SEQ ID NO: 8105), WAN00OGSP (SEQ ID NO: 8107), WAN00OGSQ (SEQ ID NO: 8108), WAN00OGS4 (SEQ ID NO: 8087), and WAN00OGT4 (SEQ ID NO: 8122).

In yet another embodiment, a protein array of the present invention includes at least one probe which can specifically bind to a protein product of a rat gene that encodes WAN00OGS4 (SEQ ID NO: 8087), and/or at least one probe which can specifically bind to a protein product of a rat gene that encodes WAN00OGT4 (SEQ ID NO: 8122).

The protein-coding sequences of rat genes can be determined using a variety of methods. Many rat protein sequences are obtainable from NCBI or other public or commercial sequence databases. The protein-coding sequences of rat genes can also be extracted from the corresponding tiling or parent sequences using an open reading frame (ORF) prediction program. Examples of ORF prediction programs include, but are not limited to, GeneMark (provided by the European Bioinformatics Institute), Glimmer (provided by The Institute for Genomic Research), and ORF Finder (provided by NCBI). Where a parent or tiling sequence represents the 5′ or 3′ untranslated region of a rat gene, a BLAST search of the sequence against a rat genome database can be conducted to determine the protein-coding region of the gene.

E. Applications

The probe arrays of the present invention can be used to detect or monitor the expression profile of rat genes. The probe arrays of the present invention can also be used to detect or evaluate agents that can modulate the expression profile of rat genes. In addition, the probe arrays of the present invention can be used to identity or validate drug targets, or to select or assess the toxicity or efficacy of drug candidates. Other applications of probe array technology, such as genotyping, protein functional analysis, or diagnosis, are also contemplated by the present invention.

Numerous protocols are available for nucleic acid array hybridization. Exemplary protocols include, but are not limited to, those provided by Affymetrix for its GeneChip® arrays. Samples amenable to nucleic acid array analysis can be prepared from any rat cell or tissue. Suitable samples include, but are not limited to, RNA samples (e.g., mRNA or cRNA) or DNA samples (e.g., cDNA).

A variety of methods can be used to isolate RNA from cells or tissues. Examples of these methods include RNeasy kits (QIAGEN Inc.), MasterPure kits (Epicentre Technologies), and TRIZOL (Gibco BRL). The RNA isolation methods provided by Affymetrix can also be used.

In many embodiments, the isolated RNA is amplified and/or labeled before being hybridized to a nucleic acid array of the present invention. Examples of RNA amplification methods include, but are not limited to, reverse transcriptase PCR, isothermal amplification, ligase chain reaction, and Qbeta replicase method. The final amplification products can be either cDNA or cRNA.

cDNA, cRNA, or other nucleic acid molecules can be labeled with one or more labeling moieties to allow for detection of hybridized polynucleotide complexes. The labeling moieties can include compositions that are detectable by spectroscopic, photochemical, biochemical, bioelectronic, immunochemical, electrical, optical, or chemical means. Suitable labeling moieties include, but are not limited to, radioisotopes, chemiluminescent compounds, labeled binding proteins, heavy metal atoms, spectroscopic markers (such as fluorescent markers or dyes), magnetic labels, linked enzymes, mass spectrometry tags, spin labels, or electron transfer donors or acceptors.

In many cases, nucleic acid molecules are fragmented before being labeled with detectable moieties. Examples of fragmentation methods include, but are not limited to, heat or ion-mediated hydrolysis.

Hybridization reactions can be performed in absolute or differential hybridization formats. In the absolute hybridization format, polynucleotides derived from one sample are hybridized to the probes in a nucleic acid array of the present invention. Signals detected after the formation of hybridization complexes correlate to the polynucleotide levels in the sample. In the differential hybridization format, polynucleotides derived from two samples are labeled with different labeling moieties. A mixture of these differently labeled polynucleotides is added to a nucleic acid array of the present invention. The nucleic acid array is then examined under conditions in which the emissions from the two different labels are individually detectable. In one embodiment, the fluorophores Cy3 and Cy5 (Amersham Pharmacia Biotech, Piscataway, N.J.) are used as the labeling moieties for the differential hybridization format.

Signals gathered from a nucleic acid array can be analyzed using commercially available software, such as those provided by Affymetrix or Agilent Technologies. Controls, such as those for scan sensitivity, probe labeling, or cDNA or cRNA quantitation, can be included in the hybridization experiments. Hybridization signals can be scaled or normalized before being further analyzed. For instance, hybridization signals for each individual probe can be normalized to take into account variations in hybridization intensities when more than one array is used under similar test conditions. Hybridization signals can also be normalized using the intensities derived from internal normalization controls contained on each array. In addition, genes with relatively consistent expression levels across the samples can be used to normalize the expression levels of other genes. In one example, probes for certain maintenance genes are included in a nucleic acid array. These genes are chosen because they show stable levels of expression across a diverse set of tissues. Hybridization signals can be normalized or scaled based on the expression levels of these maintenance genes.

In one embodiment, probes for certain exogenous transcripts are included in the nucleic acid array. These transcripts can be chosen such that they show no similarity to eukaryotic transcripts. In one example, eleven exogenous transcripts at different known concentrations are spiked into each sample. The array is first scaled to a trimmed-mean target value of 100. Based on the scaled hybridization signal of these eleven probe sets, a standard curve can be drawn such that all transcripts present in the sample can be converted from a signal value to a more meaningful concentration value. In another example, a standard curve correlating the signal value read off of the array and known frequency (molarity) can be generated when the array image is read and the probe set expression values are generated. From this standard curve, each signal value can then be converted to a “parts per million” or picomolarity value. The exogenous controls spiked into each sample can include, for instance, E. coli BioB-5, E. coli BioB-M, E. coli BioB-3, E. coli BioC-5, E. coli BioC-3, E. coli BioD-3, E. coli BioD-5, Bacteriophage P1 Cre-5, Bacteriophage P1 Cre-3, E. coli Dap-5, B. subtilis Dap-M, and B. subtilis Dap-3. Probes for these transcripts can be readily designed according to the present invention. Other suitable control sequences are depicted in Table 7.

In addition to expression profiling, the nucleic acid arrays of the present invention can also be used to detect or evaluate agents that can modulate the expression profile of rat genes. In one exemplary method, an agent of interest is first contacted with rat cells. mRNA is extracted from the cells and amplified and labeled. The amplified mRNA (e.g., cDNA or cRNA) is hybridized to a nucleic acid array of the present invention to determine if the agent can modulate the expression profile of a rat gene of interest. This can be achieved by comparing the transcript profiles before and after the treatment with the agent of interest.

Any agent of interest can be evaluated using the present invention. In one embodiment, the agent is a small molecule, an antibody, a toxin (including a recombinant immunotoxin), a substrate or pseudosubstrate recognizable by a protein product of a rat gene, or a naturally-occurring factor or an analog thereof. Examples of naturally-occurring factors include, but are not limited to, endocrine factors, paracrine factors, autocrine factors, intracellular factors, and factors interacting with cell receptors. In another embodiment, the agent is an antisense RNA, a double stranded RNA having RNA interference (RNAi) effect, or a vector encoding an antisense or RNAi sequence. Once a lead agent is identified, its derivatives or analogs can be further screened or tested for the optimal modulatory effect.

Any in vitro or in vivo assay format can be used to detect or evaluate modulators of rat genes. Suitable assay formats include, but are not limited to, in vitro transcription and translation, cultured cell lines, primary cell cultures, or tissue cultures. In one embodiment, high-throughput screen assays or compound libraries are employed for the identification of desired modulators.

The modulatory effect of an agent can be further detected or evaluated in rat. In an exemplary method, an agent of interest is first administered to a rat. A nucleic acid sample is prepared from the rat and hybridized to a nucleic acid array of the present invention. Hybridization signals are then analyzed to determine if the agent can modulate the expression profile of a rat gene in a desired manner.

In many embodiments, the rat genes being investigated are rat orthologs or homologs of human drug target or disease genes. Examples of drug target genes include, but are not limited to, kinase genes, phosphatase genes, protease genes, G-protein coupled receptor genes, nuclear hormone receptor genes, or ion channel genes. Examples of disease genes include, but are not limited to, those that are differentially expressed in diseased tissues as compared to the corresponding healthy tissues.

The nucleic acid arrays of the present invention can also be used to assess the specificity or toxicity of a drug candidate. An ideal drug candidate modulates only the specified rat gene(s) without significantly affecting the expression or function of other rat genes. The nucleic acid arrays of the present invention allow for the identification of compounds that only modulate a particular rat gene or genes.

Furthermore, the nucleic acid arrays of the present invention can be used to investigate drug-drug interactions. Simultaneous administration of several drugs is often necessary to achieve desired therapeutic objectives. For instance, in cancer chemotherapy, antimicrobial therapy or AIDS treatment, drug combination is usually desirable in order to delay the emergence of drug resistant tumor cells, microbes or viruses. However, drug combination may also cause unexpected adverse effects. These adverse effects can be the result of an unintended activation or suppression of certain signaling pathways. The expression profile of each component in these signaling pathways can be monitored using a nucleic acid array of the present invention to determine if a drug combination can produce any unintended effect on these pathways.

The hybridization data generated from the nucleic acid arrays of the present invention can be stored in a database for future analysis. This database can be used as an informational translator that takes information on a gene directly to a compound that has been found to affect the expression of that gene. For instance, if the database reveals that compound X alters the expression of gene Y, and a paper is published reporting that the expression of gene Y is sensitive to a particular signal transduction pathway, then compound X becomes a candidate for modulating that signal transduction pathway. This effectively leverages the value of the publicly available data on the identification of potential drug candidates.

The same instrumentation as used for nucleic acid array analysis is readily applicable to the protein arrays of the present invention. Many genes have alternatively spliced isoforms, which may have different functions. Post-translational modifications also give protein variations. The protein arrays of the present invention allow the detection or assessment of one specific form of a protein and therefore enable drug target validation at the proteomics level.

The agents identified in the present invention can be used to treat patients who have a disease caused by abnormal expression of one or more disease genes. An agent that modulates the expression of these disease genes can be administered to a patient in need thereof. Any method known in the art may be used to administer a desired agent to a patient of interest.

The present invention further contemplates polynucleotide or polypeptide collections. In one embodiment, a polynucleotide collection of the present invention comprises at least 1, 2, 5, 10, 50, 100, 500, 1,000, 2,000, 3,000, 4,000, or more probes, and each of these probes is capable of hybridizing under stringent or nucleic acid array hybridization conditions to a respective tiling sequence selected from SEQ ID NOs: 4,097-8,192, or the complement thereof. In another embodiment, a polynucleotide collection of the present invention comprises at least one sequence selected from SEQ ID NOs: 1-8,192, or the complement thereof. In still another embodiment, a polypeptide collection of the present invention includes at least 1, 2, 5, 10, 50, 100, 500, 1,000, or more polypeptides, each of which is a protein product of a respective rat gene that encodes a sequence selected from SEQ ID NOs: 1-8,192.

It should be understood that the above-described embodiments and the following examples are given by way of illustration, not limitation. Various changes and modifications within the scope of the present invention will become apparent to those skilled in the art from the present description.

### F. EXAMPLES

### Example 1

### Nucleic Acid Array

The tiling sequences depicted in SEQ ID NOs: 4,097-8,192 were submitted to Affymetrix for custom array design. Affymetrix selected probes for each tiling sequence using its probe-picking algorithm. Non-ambiguous probes with 25 bases in length were selected. Forty-six probe-pairs were requested for each tiling sequence with a minimum number of acceptable probe-pairs set to twenty-five. The final array was directed to 3,964 rat transcripts and 120 endogenous and exogenous control probes sets. The perfect match probes on the final array are depicted in SEQ ID NOs: 174,983-362,830. The qualifiers of these probes are illustrated in Table 8.

### Example 2

### Nucleic Acid Array Hybridization

10 μg of biotin-labeled sample DNA/RNA is diluted in 1×MES buffer with 100 μg/ml herring sperm DNA and 50 μg/ml acetylated BSA. To normalize arrays to each other and to estimate the sensitivity of the nucleic acid arrays, in vitro synthesized transcripts of control genes are included in each hybridization reaction. The abundance of these transcripts can range from 1:300,000 (3 ppm) to 1:1000 (1000 ppm) stated in terms of the number of control transcripts per total transcripts. As determined by the signal response from these control transcripts, the sensitivity of detection of the arrays can range, for example, between about 1:300,000 and 1:100,000 copies/million. Labeled DNA/RNA are denatured at 99° C. for 5 minutes and then 45° C. for 5 minutes and hybridized to the nucleic array of Example 1. The array is hybridized for 16 hours at 45° C. The hybridization buffer includes 100 mM MES, 1 M [Na⁺], 20 mM EDTA, and 0.01% Tween 20. After hybridization, the cartridge(s) is washed extensively with wash buffer (6×SSPET), for instance, three 10-minute washes at room temperature. The washed cartridge(s) is then stained with phycoerythrin coupled to streptavidin.

12×MES stock contains 1.22 M MES and 0.89 M [Na⁺]. For 1000 ml, the stock can be prepared by mixing 70.4 g MES free acid monohydrate, 193.3 g MES sodium salt and 800 ml of molecular biology grade water, and adjusting volume to 1000 ml. The pH should be between 6.5 and 6.7. 2×hybridization buffer can be prepared by mixing 8.3 ml of 12×MES stock, 17.7 ml of 5 M NaCl, 4.0 ml of 0.5 M EDTA, 0.1 ml of 10% Tween 20 and 19.9 ml of water. 6×SSPET contains 0.9 M NaCl, 60 mM NaH₂PO₄, 6 mM EDTA, pH 7.4, and 0.005% Triton X-100. In some cases, the wash buffer can be replaced with a more stringent wash buffer. 1000 ml stringent wash buffer can be prepared by mixing 83.3 ml of 12×MES stock, 5.2 ml of 5 M NaCl, 1.0 ml of 10% Tween 20 and 910.5 ml of water.

The foregoing description of the present invention provides illustration and description, but is not intended to be exhaustive or to limit the invention to the precise one disclosed. Modifications and variations are possible consistent with the above teachings or may be acquired from practice of the invention. Thus, it is noted that the scope of the invention is defined by the claims and their equivalents.

1. A nucleic acid array comprising at least one polynucleotide which is capable of hybridizing under stringent or nucleic acid array hybridization conditions to a rat gene that encodes a sequence selected from the group consisting of SEQ ID NOs: 7622 and 8084-8124.

2. The nucleic acid array of claim 1, comprising at least five polynucleotides, each of which is capable of hybridizing under stringent or nucleic acid array hybridization conditions to a different respective rat gene that encodes a sequence selected from the group consisting of SEQ ID NOs: 7622 and 8084-8124.

3. The nucleic acid array of claim 1, comprising at least ten polynucleotides, each of which is capable of hybridizing under stringent or nucleic acid array hybridization conditions to a different respective rat gene that encodes a sequence selected from the group consisting of SEQ ID NOs: 7622 and 8084-8124.

4. The nucleic acid array of claim 1, wherein said polynucleotide is capable of hybridizing under stringent or nucleic acid array hybridization conditions to a sequence selected from the group consisting of SEQ ID NOs: 7622 and 8084-8124, or the complement thereof.

5. The nucleic acid array of claim 1, comprising at least five polynucleotides, each of which is capable of hybridizing under stringent or nucleic acid array hybridization conditions to a different respective sequence selected from the group consisting of SEQ ID NOs: 7622 and 8084-8124, or the complement thereof.

6. The nucleic acid array of claim 1, comprising at least ten polynucleotides, each of which is capable of hybridizing under stringent or nucleic acid array hybridization conditions to a different respective sequence selected from the group consisting of SEQ ID NOs: 7622 and 8084-8124, or the complement thereof.

7. The nucleic acid array of claim 1, comprising at least 42 polynucleotides, each of which is capable of hybridizing under stringent or nucleic acid array hybridization conditions to a different respective sequence selected from the group consisting of SEQ ID NOs: 7622 and 8084-8124.

8. The nucleic acid array of claim 1, comprising at least 42 polynucleotides, each of which is capable of hybridizing under stringent or nucleic acid array hybridization conditions to the complement of a different respective sequence selected from the group consisting of SEQ ID NOs: 7622 and 8084-8124.

9. The nucleic acid array of claim 1, wherein said polynucleotide is capable of hybridizing under stringent or nucleic acid array hybridization conditions to a sequence selected from the group consisting of WAN00OGR4 (SEQ ID NO: 8084), WAN00OGSD (SEQ ID NO: 8095), WAN00OGSE (SEQ ID NO: 8096), WAN00OGSH (SEQ ID NO: 8099), WAN00OGSK (SEQ ID NO: 8102), WAN00OGSN (SEQ ID NO: 8105), WAN00OGSP (SEQ ID NO: 8107), WAN00OGSQ (SEQ ID NO: 8108), WAN00OGS4 (SEQ ID NO: 8087) and WAN00OGT4 (SEQ ID NO: 8122), or the complement of said sequence.

10. The nucleic acid array of claim 1, wherein said polynucleotide is capable of hybridizing under stringent or nucleic acid array hybridization conditions to a sequence selected from the group consisting of WAN00OGS4 (SEQ ID NO: 8087) and WAN00OGT4 (SEQ ID NO: 8122), or the complement of said sequence.

11. The nucleic acid array of claim 1, comprising at least 100 polynucleotides, each of which is capable of hybridizing under stringent or nucleic acid array hybridization conditions to a different respective tiling sequence selected from SEQ ID NOs: 4,097-8,192, or the complement thereof.

12. The nucleic acid array of claim 1, comprising at least 1,000 polynucleotides, each of which is capable of hybridizing under stringent or nucleic acid array hybridization conditions to a different respective tiling sequence selected from SEQ ID NOs: 4,097-8,192, or the complement thereof.

13. The nucleic acid array of claim 1, wherein a substantial portion of all polynucleotides that are stably attached to the nucleic acid array is probes for rat genes.

14. A method for expression profiling of rat genes, comprising:
preparing a nucleic acid sample from rat cells; and
hybridizing said nucleic acid sample to the nucleic acid array of claim 13.

15. A method for identifying or evaluating an agent capable of modulating gene expression in rat cells, comprising:
contacting said agent with said rat cells;
preparing a nucleic acid sample from said rat cells; and
hybridizing the nucleic acid sample to the nucleic acid array of claim 13,
wherein a change in gene expression profile in said rat cells after said contacting, as compared to before said contacting, is indicative that said agent is capable of modulating gene expression in said rat cells.

16. The method of claim 15, wherein said agent modulates the expression of a rat gene in said rat cells, and said rat gene is an ortholog of a human drug target gene or a human disease gene.

17. A probe array comprising probes for rat genes, wherein said rat genes include at least one gene which encodes a sequence selected from the group consisting of SEQ ID NOs: 7622 and 8084-8124.

18. The probe array of claim 17, wherein a substantial portion of all probes that are stably attached to the probe array is antibodies that are specific for the protein products of said rat genes.

19. The probe array of claim 17, wherein said probes include an antibody which is specific for a protein product of a rat gene that encodes a sequence selected from the group consisting of WAN00OGR4 (SEQ ID NO: 8084), WAN00OGSD (SEQ ID NO: 8095), WAN00OGSE (SEQ ID NO: 8096), WAN00OGSH (SEQ ID NO: 8099), WAN00OGSK (SEQ ID NO: 8102), WAN00OGSN (SEQ ID NO: 8105), WAN00OGSP (SEQ ID NO: 8107), WAN00OGSQ (SEQ ID NO: 8108), WAN00OGS4 (SEQ ID NO: 8087), and WAN00OGT4 (SEQ ID NO: 8122).

20. A biomolecule collection comprising:
(1) at least one isolated polynucleotide comprising a sequence selected from SEQ ID NOs: 1-8,192, or the complement thereof;
(2) at least one isolated polypeptide product of a rat gene which encodes a sequence selected from SEQ ID NOs: 1-8,192; or
(3) at least one antibody specifically recognizing said polypeptide product.
